(19) United States
(12) Patent Application Publication
(10) Pub. No.: US 2002/0177817 A1
(43) Pub. Date: Nov. 28, 2002

(54) Intravenous tubing cuff

(76) Inventor: Machelle Daniels, Trabuco Canyon, CA (US)

Correspondence Address: Kenneth W. Float, The Law Offices of Kenneth W. Float, P.O. Box 80790, Rancho Santa Margarita, CA, 92688, US

(21) Appl. No.: 10/092,067

(22) Filed: Mar. 5, 2002

Related U.S. Application Data
(60) Provisional application No. 60/293,311, filed on May 25, 2001.

(51) Int. Cl. A61M 5/32

(52) U.S. Cl. 604/179

(57) ABSTRACT

A disposable noninvasive tubing cuff for use in securing an I.V. tube, or the like, to a patient. The disposable I.V. tubing cuff is removably secured to a patient's limb, such as a wrist arm or leg, and holds the I.V. tube without sliding on the patient's limb. An exemplary cuff comprises a layer of soft, porous, cloth-like material with a thin layer of soft non-skid porous foam rubber secured to one side thereof that touches the patient's skin. Opposite ends of the cuff are secured together using hook and loop materials, wherein hooks contact and engage loops to hold opposite ends of the cuff together. A bendable or foldable adhesive layer is attached to the porous, cloth-like material on the same side as the layer of loop material, and which has a protective layer that covers adhesive which is removed to expose the adhesive. A tube is laid on the exposed adhesive and the adhesive layer is wrapped around the tube to secure it to the cuff.

### BACKGROUND OF THE INVENTION

The present invention relates generally to apparatus for securing an intravenous (I.V.) tube to a patient, and more particularly, to a disposable noninvasive intravenous (I.V.) tubing cuff that is used for such purposes.

A conventional way to attach or secure an I.V. tube to a patient is as follows. From the insertion site, the I.V. tubing is formed into a short loop to the left or right of the insertion site, as applicable, and secured in place with short lengths of adhesive tape. The tubing is then laid out along the length of the body, wrist, leg, etc., and secured with additional lengths of adhesive tape. In certain patients having thin skin, for example, removal of the tape tears or otherwise impairs the integrity of the skin, subjecting patients to subsequent infections, which necessitate additional treatment. Hypoallergenic components prevent allergic rashes and irritation which are commonly experienced with the application of conventional adhesive tape in securing I.V. tubing to skin. Valuable time is saved by avoiding the use of varied strips of adhesive tape to secure tubing to skin. Tape must frequently be replaced which exponentially increases these problems.

As the patient moves, the tubing catches on bedding, equipment, or the patient rolls over on it. Each time this happens the tube is jerked, and the insertion site, despite the adhesive tape, is jerked at as well. Anyone who has had the experience of an I.V. is familiar with the pain and distress associated with this constant tug of war.

In addition to the pain, trauma is exerted on the insertion vein, often leading to vascular spasm at the insertion site. T he vein collapses at the site, necessitating removal of the I.V. which is then inserted at a new site. This results in additional patient misery.

### SUMMARY OF THE INVENTION

The present invention is a single-patient disposable noninvasive intravenous (I.V.) tubing cuff for use in securing an I.V. tube, or the like, to a patient. The disposable I.V. tubing cuff is removably secured to a patient's limb, such as a wrist arm or leg, and holds the I.V. tube without sliding on the patient's limb. The disposable I.V. tubing cuff provides for an improvement over the use of adhesive tape to secure I.V. tube to a patient's limb.

The disposable I.V. tubing cuff is a disposable medical device having a self-adjusting intravenous tubing cuff configuration. The device is constructed using hypoallergenic components, secures and stabilizes I.V. tubing to the patient's limb without adhesive material. The device thus preserves skin integrity and prevents dislodging of the I.V. tubing.

An exemplary disposable I.V. cuff comprises a strip or layer of soft, porous, cloth-like material. A thin strip or layer of soft “non-skid” porous foam rubber is secured to one side of the layer of porous, cloth-like material. The layer of soft porous foam rubber touches the patient's skin.

The porous nature of both the cloth-like material and the foam rubber permit the cuff to breath and allow oxygen to get to the patient's skin. The layer of foam rubber prevents the cuff from rolling around the limb or sliding up and down the limb.

Opposite ends of the cuff are secured together using hook and loop materials, such as Velcro™ material, for example. A piece or layer of hook material having hooks thereon is attached or secured to one end of the porous, cloth-like material on the same side and adjacent to the layer of porous, foam material.

A layer of loop material is attached or secured to the opposite end of the porous, cloth-like material on a side that is opposite to the hook material. When the cuff is wrapped around the patient's limb, hooks of the hook material come into contact with and engage loops of the loop material to hold the two ends of the cuff together.

A bendable or foldable adhesive layer is attached or secured to the porous, cloth-like material on the same side as the layer of loop material. The adhesive layer preferably has a protective layer that covers adhesive which is removed to expose the adhesive. An I.V. is laid on the exposed adhesive and the adhesive layer is wrapped around the I.V. to secure it to the cuff.

### BRIEF DESCRIPTION OF THE DRAWINGS

The various features and advantages of the present invention may be more readily understood with reference to the following detailed description taken in conjunction with the accompanying drawings, wherein like reference numerals designate like structural elements, and in which:

FIG. 1 is a top view of an exemplary I.V. cuff in accordance with the principles of the present invention;

FIG. 2 is a side view of the exemplary I.V. cuff;

FIG. 3 is a bottom view of the exemplary I.V. cuff;

FIG. 4 is a view of the exemplary I.V. cuff disposed on a person's wrist looking at the inside of the wrist;

FIG. 5 is a view of the exemplary I.V. cuff disposed on a person's wrist looking at the outside of the wrist; and

FIG. 6 is a view of the exemplary I.V. cuff disposed on a person's wrist looking at the outside of the wrist and showing attachment of an I.V. tubing.

### DETAILED DESCRIPTION

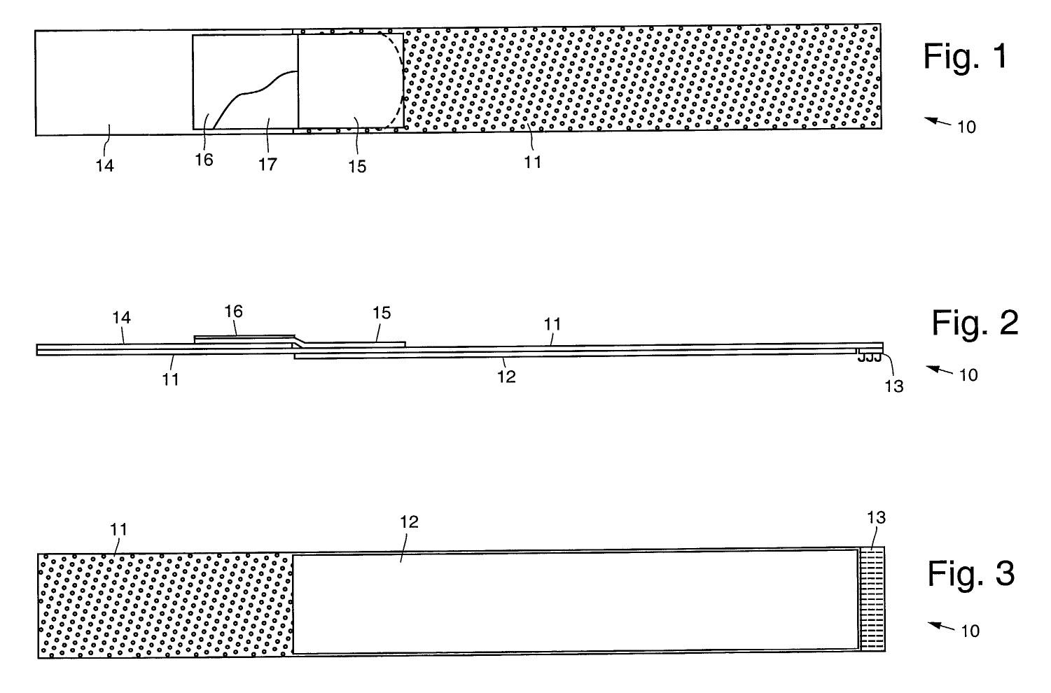

Referring to the drawing figures, FIGS. 1-3 show top, side, and bottom view, respectively, of an exemplary I.V. cuff 10 in accordance with the principles of the present invention. The disposable I.V. cuff 10 comprises a strip or layer of soft, porous, cloth-like material 11. The strip or layer of soft, porous, cloth-like material 11 may be a micro porous film 11 available from 3M Corporation, St. Paul Minn., for example.

The microporous film 11 is a polypropylene material that is breathable and is resistant to liquid transmission and very small particles. The microporous film 11 is soft, and hypoallergenic. A derivative of this film 11 is a Propore™ fabric available from 3M Corporation, which is a laminate of the microporous film to a polypropylene nonwoven material to provide strength and durability.

A thin strip or layer of “non-skid” porous foam rubber 12 is secured to one side of the layer of porous, cloth-like material 11. The layer of porous foam rubber 12 is placed against the patient's skin. The soft, porous foam rubber 12 may be obtained from Rogers Foam Corporation, Somerville, Mass., for example.

The porous nature of both the cloth-like material 11 and the foam rubber 12 permit the cuff to breath and allow oxygen to get to the patient's skin. The layer of foam rubber 12 prevents the cuff 10 from rolling around the limb, sliding up and down the limb, or dislodging the I.V.

Opposite ends of the cuff 10 are secured together using a reclosable fastener employing hook and loop materials 13, 14, such as Velcro™ material, or a loop and hook system, available from 3M Corporation, for example. The loop and hook system has a nonwoven loop with a film backing. The hooks are attached to a durable backing layer. A knitted loop and hook system is also available from 3M Corporation. The knitted loop and hook system has a adhesive coated, liner-less loop that allows adhesion to the porous, cloth-like material 11.

A piece or layer of hook material 13 having hooks thereon is attached or secured to one end of the porous, cloth-like material 11 on the same side and adjacent to the layer of porous foam rubber 12. A layer of loop material 14 is attached or secured to the opposite end of the porous, cloth-like material 11 on a side that is opposite to the hook material 13. When the cuff 10 is wrapped around the patient's limb, hooks of the hook material 13 come into contact with and engage loops of the loop material 14 to hold the two ends of the cuff 10 together.

A bendable or foldable adhesive layer 15 (comprising an adhesive tab 15 on one end thereof, which may be curved as illustrated by the dashed line in FIG. 1) is attached or secured to the porous, cloth-like material 11 on the same side as the layer of loop material 14. The adhesive layer 15 preferably has a protective layer 16 that covers adhesive material 17 which is removed to expose the adhesive material 17 of the adhesive layer 15. An I.V. tube 23 having an I.V. 21 attached to its end (FIG. 6) is laid on the exposed adhesive 17 and the adhesive tab 15 is wrapped around the I.V. tube 23 to secure it and the I.V. 21 to the cuff 10. The adhesive tab 15 is wrapped around the I.V. tube 23 to provide proper and secure placement of the I.V. tube 23 and I.V. 21 on the patient.

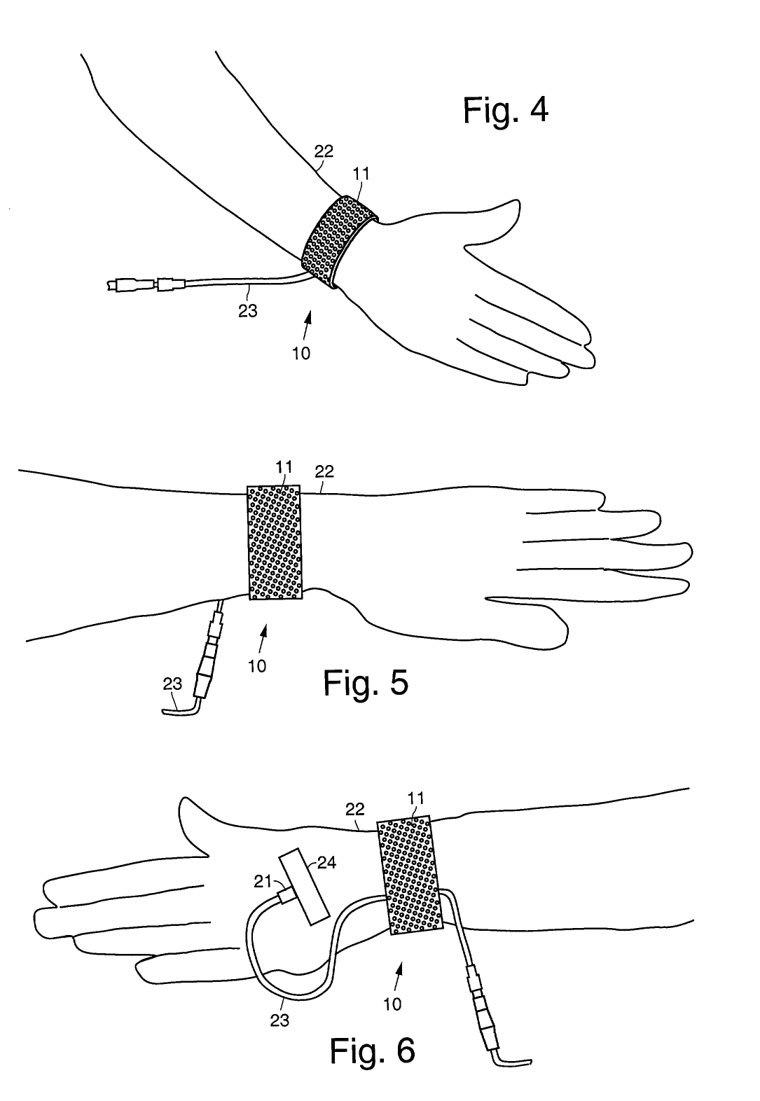

FIGS. 4-6 are various views of the exemplary I.V. cuff 10 disposed on a patient's wrist 22 and showing attachment of an I.V. tube 23. As is seen in FIGS. 4-6, the I.V. cuff 10 simply and effectively secures the I.V. tube 23 to a wrist 22 (or other limb 22) of a patient. In FIG. 6, the I.V. 21 is shown inserted into the patient and is secured in place using adhesive tape 24, for example.

The I.V. cuff 10 is inexpensive, and all materials used are readily available and currently in use on other inexpensive products. The I.V. cuff 10 is easy to apply, and may be applied to one hand, left or right.

The I.V. cuff 10 remains in position under even excessive strain. The thin layer of “non-skid” foam rubber 12 lays across the top of the wrist 22 under the I.V. 21 to about half way down on each side. This effectively prevents the I.V. cuff 10 from either rolling around the wrist 22 or sliding up and down the arm, for example.

The I.V. cuff 10 prevents inordinate tugging at the insertion site. The I.V. cuff 10 is fastened with Velcro-type or hook-and-loop material 13, 14, to a comfortable but firm pressure, and is wide enough to buffer the usual amount of jerking, tugging, and pulling. The I.V. cuff 10 is adjustable using the Velcro-type or hook-and-loop material 13, 14, to provide the maximum amount of comfort for the individual patient.

The I.V. cuff 10 is extremely comfortable. The I.V. cuff 10 is made of soft, strong, cloth-like material 11, while the foam 12 underside is soft and cushion the skin. Both materials are very thin and porous. The skin can breathe and will not sweat under the I.V. cuff 10, and the I.V. cuff 10 dries quickly if it gets wet.

The I.V. cuff 10 has a number of advantages over the use of adhesive tape, for example. The I.V. cuff 10 is noninvasive and does not irritate the skin. The I.V. cuff 10 is hypo-allergenic. The I.V. cuff 10 is easy to use and is quickly applied. The I.V. cuff 10 provides for patient comfort in that no tape irritates the skin, it decreases possible infection and/or bruising, it decrease infiltration, it decreases “blown” I,V.'s 21, and decreases the number of I.V. restarts.

The I.V. tubing 23 is held securely in place using the I.V. cuff 10. In contrast, adhesive tape frequently does not hold (due to moisture, oil, hair, etc.). The I.V. cuff 10 assures a secure hold to the patient's limb 22 and a perfect fit in each application. The adhesive tabs 15 (adhesive layer 15) that secure the I.V. tubing 23 may be reused as needed. The I.V. cuff 10 may be sized for use in adult, pediatric and infant applications. The I.V. cuff 10 may be color coded, related to the application for easy recognition (as are needles and syringes).

The I.V. cuff 10 may be used in hospitals, and acute care, convalescent, and hospice facilities, out-patient clinics, dentistry and oral surgery offices, rescue or medivac transport applications, fire and rescue paramedics. The I.V. cuff 10 may be modified for use in veterinary medicine applications.

Thus, a disposable noninvasive intravenous (I.V.) tubing cuff for securing an I.V. tube to a patient has been disclosed. It is to be understood that the described embodiment is merely illustrative of some of the many specific embodiments that represent applications of the principles of the present invention. Clearly, numerous and other arrangements can be readily devised by those skilled in the art without departing from the scope of the invention.

What is claimed is:

1. A tubing cuff for securing a tube to a limb of a patient, comprising:
a layer of porous, cloth-like material;
a layer of porous foam rubber secured to one side of the layer of porous, cloth-like material;
a reclosable fastener for securing distal ends of the cuff together; and
a bendable adhesive layer having an adhesive attached to the porous, cloth-like material on the same side as the layer of loop material.

2. The tubing cuff recited in claim 1 wherein the layer of porous, cloth-like material comprises microporous film.

3. The tubing cuff recited in claim 2 wherein the microporous film comprises polypropylene material.

4. The tubing cuff recited in claim 2 wherein the microporous film is hypoallergenic.

5. The tubing cuff recited in claim 2 wherein the microporous film comprises a laminate of microporous film and a polypropylene nonwoven material

6. The tubing cuff recited in claim 1 wherein the layer of porous, cloth-like material is hypoallergenic.

7. The tubing cuff recited in claim 1 wherein the layer of porous foam rubber comprises non-skid porous foam rubber.

8. The tubing cuff recited in claim 1 wherein the reclosable fastener comprises hook and loop materials.

9. The tubing cuff recited in claim 8 wherein the hook and loop materials comprise a nonwoven loop with a film backing.

10. The tubing cuff recited in claim 8 wherein the hooks are attached to a durable backing layer.

11. The tubing cuff recited in claim 8 wherein the reclosable fastener comprises knitted loop and hook materials.

12. The tubing cuff recited in claim 1 wherein the reclosable fastener comprises
a layer of hook material having hooks thereon attached to one end of the porous, cloth-like material on the same side and adjacent to the layer of porous foam rubber; and
a layer of loop material attached to an opposite end of the porous, cloth-like material on a side that is opposite to the hook material.

13. The tubing cuff recited in claim 1 wherein the adhesive layer has a protective layer that covers adhesive material and which is removed to expose the adhesive material of the adhesive layer.

14. The tubing cuff recited in claim 1 wherein the layer of porous foam rubber is placed against the patient's limb and secured by the reclosable fastener, and wherein a tube is laid on exposed adhesive of the bendable adhesive layer, which bendable adhesive layer is wrapped around the tube to secure it to the cuff.
